SEQUENCE LISTING (16 sequences)

SEQ ID NO: 1 - DNA, 29 bp, artificial sequence
  primer NcoI exons 1-28 from the human gene for dysferlin
ttccatgggcatgctgagggtcttcatcc

SEQ ID NO: 2 - DNA, 42 bp, artificial sequence
  primer HindIII MfeI exons 1-28 from the human gene for dysferlin
ttcaattgggaagcttgcccaccttgctcatcgacagcccgg

SEQ ID NO: 3 - DNA, 40 bp, artificial sequence
  primer SpeI MluI exon 29-55 from the human gene for dysferlin
ttactagtggacgcgtccaggctgggagtatagcatcacc

SEQ ID NO: 4 - DNA, 45 bp, artificial sequence
  primer NotI exon 29-55 from the human gene for dysferlin
ttgcggccgcctacagggcaggagagtcctcagctgaagggcttc

SEQ ID NO: 5 - DNA, 24 bp, artificial sequence
  primer HindIII-SD5′ from the human gene for dysferlin
ttaagcttagcatgtggaacctgg

SEQ ID NO: 6 - DNA, 27 bp, artificial sequence
  primer MfeI-SD3′ from the human gene for dysferlin
ttcaattgagcttggagtggggggtgc

SEQ ID NO: 7 - DNA, 30 bp, artificial sequence
  primer SpeI-SA5′ from the human gene for dysferlin
ttactagtgcaaattaggaccgagagtcag

SEQ ID NO: 8 - DNA, 28 bp, artificial sequence
  primer MluI-SA from the human gene for dysferlin
ttacgcgtgggagggggaaccggtcact

SEQ ID NO: 9 - DNA, 18 bp, artificial sequence
  primer Exon28.f from the human gene for dysferlin
ctcaaccgggctgtcgat

SEQ ID NO: 10 - DNA, 26 bp, artificial sequence
  primer Exon29.r from the human gene for dysferlin
gtcggtgtgtgtagtacatcttctca

SEQ ID NO: 11 - DNA, 12 bp, artificial sequence
  primer Exons2829.s from the human gene for dysferlin
caaggctgggag

SEQ ID NO: 12 - DNA, 264 bp, artificial sequence
  intron 28 of the human gene for dysferlin
gtgggcagcatgtggaacctggcgagccccatccccggcaagctctcaagccatgctggtggggacgactgaatgccagggcccttcactgggctatttcacccagggacgcttcttgaaggcaccccccactccaagctgcaaattaggaccgagagtcagtggccgctcaagagtctgtgaccatgccccaaattcagagatggtcccaggagagatggggggaactgccaagcaatgagtgaccggttccccctcccccag

SEQ ID NO: 13 - DNA, 275 bp, artificial sequence
  intron 28 modified from the human gene for dysferlin
gtgggcaagcttagcatgtggaacctggcgagccccatccccggcaagctctcaagccatgctggtggggacgactgaatgccagggcccttcactgggctatttcacccagggacgcttcttgaaggcaccccccactccaagctcaaattaggaccgagagtcagtggccgctcaagagtctgtgaccatgccccaaattcagagatggtcccaggagagatggggggaactgccaagcaatgagtgaccggttccccctcccacgcgtccag

SEQ ID NO: 14 - DNA, 146 bp, artificial sequence
  intron 28 modified vector 1 from the human gene for dysferlin
gtgggcaagcttagcatgtggaacctggcgagccccatccccggcaagctctcaagccatgctggtggggacgactgaatgccagggcccttcactgggctatttcacccagggacgcttcttgaaggcaccccccactccaagct

SEQ ID NO: 15 - DNA, 129 bp, artificial sequence
  intron 28 modified vector 2 from the human gene for dysferlin
caaattaggaccgagagtcagtggccgctcaagagtctgtgaccatgccccaaattcagagatggtcccaggagagatggggggaactgccaagcaatgagtgaccggttccccctcccacgcgtccag

SEQ ID NO: 16 - protein, 2080 aa, homo sapiens
  human dysferlin
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEFRMDVGTIYREPRHAYLRKWLLLSDPDDFSAGARGYLKTSLCVLGPGDEAPLERKDPSEDKEDIESNLLRPTGVALRGAHFCLKVFRAEDLPQMDDAVMDNVKQIFGFESNKKNLVDPFVEVSFAGKMLCSKILEKTANPQWNQNITLPAMFPSMCEKMRIRIIDWDRLTHNDIVATTYLSMSKISAPGGEIEEEPAGAVKPSKASDLDDYLGFLPTFGPCYINLYGSPREFTGFPDPYTELNTGKGEGVAYRGRLLLSLETKLVEHSEQKVEDLPADDILRVEKYLRRRKYSLFAAFYSATMLQDVDDAIQFEVSIGNYGNKFDMTCLPLASTTQYSRAVFDGCHYYYLPWGNVKPVVVLSSYWEDISHRIETQNQLLGIADRLEAGLEQVHLALKAQCSTEDVDSLVAQLTDELIAGCSQPLGDIHETPSATHLDQYLYQLRTHHLSQITEAALALKLGHSELPAALEQAEDWLLRLRALAEEPQNSLPDIVIWMLQGDKRVAYQRVPAHQVLFSRRGANYCGKNCGKLQTIFLKYPMEKVPGARMPVQIRVKLWFGLSVDEKEFNQFAEGKLSVFAETYENETKLALVGNWGTTGLTYPKFSDVTGKIKLPKDSFRPSAGWTWAGDWFVCPEKTLLHDMDAGHLSFVEEVFENQTRLPGGQWIYMSDNYTDVNGEKVLPKDDIECPLGWKWEDEEWSTDLNRAVDEQGWEYSITIPPERKPKHWVPAEKMYYTHRRRRWVRLRRRDLSQMEALKRHRQAEAEGEGWEYASLFGWKFHLEYRKTDAFRRRRWRRRMEPLEKTGPAAVFALEGALGGVMDDKSEDSMSVSTLSFGVNRPTISCIFDYGNRYHLRCYMYQARDLAAMDKDSFSDPYAIVSFLHQSQKTVVVKNTLNPTWDQTLIFYEIEIFGEPATVAEQPPSIVVELYDHDTYGADEFMGRCICQPSLERMPRLAWFPLTRGSQPSGELLASFELIQREKPAIHHIPGFEVQETSRILDESEDTDLPYPPPQREANIYMVPQNIKPALQRTAIEILAWGLRNMKSYQLANISSPSLVVECGGQTVQSCVIRNLRKNPNFDICTLFMEVMLPREELYCPPITVKVIDNRQFGRRPVVGQCTIRSLESFLCDPYSAESPSPQGGPDDVSLLSPGEDVLIDIDDKEPLIPIQEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

(19) United States
(12) Patent Application Publication
(10) Pub. No.: US 2010/0266551 A1
(43) Pub. Date: Oct. 21, 2010

(54) ADENO-ASSOCIATED VIRAL VECTORS FOR THE EXPRESSION OF DYSFERLIN

(75) Inventors: Isabelle Richard, Corbeil Essonnes (FR); Marc Bartoli, Paris (FR)

Correspondence Address: Leason Ellis LLP, 81 Main Street, Suite 503, White Plains, NY, 10601, US

(73) Assignees: GENETHON, Evry (FR); CENTRE NATIONAL DE LA RECHERCHE SCIENTIFIQUE, Paris (FR)

(21) Appl. No.: 12/670,451

(22) PCT Filed: Jul. 25, 2008
(86) PCT No.: PCT/FR08/51414

§ 371 Date: Jun. 24, 2010

(30) Foreign Application Priority Data
Jul. 26, 2007 (FR) 0705479

(51) Int. Cl. A61K 35/76; C12N 15/63; C12P 21/00; A61P 19/00; A61P 21/00

(52) U.S. Cl. 424/93.6; 435/320.1; 435/69.1

(57) ABSTRACT

The present invention relates to a composition comprising: a first adeno-associated viral (AAV) vector comprising: i) a 5′ITR (Inverted Terminal Repeat) sequence of AAV; ii) a portion of gene placed under the control of a promoter; iii) a sequence comprising a splice donor site; iv) a 3′ITR sequence of AAV; and/or a second adeno-associated viral (AAV) vector comprising; v) a 5′ITR (Inverted Terminal Repeat) sequence of AAV; vi) a sequence comprising a splice acceptor site; vii) a portion of gene; viii) a 3′ITR sequence of AAV. The combination of the portions of gene carried by the first and second AAV vectors comprises an open reading frame which encodes a functional dysferlin. In addition, the combination of the sequence comprising the splice donor site and the sequence comprising the splice acceptor site contains all the elements necessary for the splicing, advantageously derived from a natural intron of the dysferlin gene.

### TECHNICAL FIELD

This invention concerns the treatment of dysferlinopathies.

It calls for the use of two recombinant AAV vectors in order to express functional dysferlin. It has the advantage of being based on the native sequences of the dysferlin to gene and of being effective.

### PRIOR ART

Dysferlinopathies are due to mutations in the DYSF gene encoding the protein is dysferlin and include three clinically distinct diseases (9) (17):
Type 2B Limb Girdle Muscular Dystrophy (LGMD2B; OMIM 253601);
Miyoshi Myopathy (MK OMIM 254130); and
Distal Anterior Compartment Myopathy (DACM; OMIM 606768).

The first two phenotypes are characterised by increased levels of creatine kinase and the slow progression of muscle weakness. In LGMD2B, the proximal muscles of the limbs and trunk are most affected whereas in MM it is the distal muscles of the lower limbs that are involved. DACM is initially distal, with the anterior part of the muscles being affected. Progression of the disease is rapid causing severe weakness of the proximal muscles.

All the possible types of mutations have been identified in the DYSF gene, including missense and nonsense mutations, deletions or insertions, splicing mutations and large deletions (5)(6)(11)(15).

However, major polymorphism is also present in the gene. Recent studies indicate that, in most cases, the loss of “visible” dysferlin is associated with a nonsense mutation or loss of the reading frame (15).

In humans, DYSF is localised in the chromosomal region 2p13 and belongs to the large gene category, since it is composed of 55 exons spread over 150 kb of genomic DNA and is transcribed into a 6.5 kb messenger (5) (11).

Although expressed in various tissues such as the brain and the lungs, the DYSF gene is mainly expressed in skeletal muscle, the heart and monocytes/macrophages (2).

Dysferlin is a 237 kDa protein composed of 2080 amino acids, including a C-terminal transmembrane domain and a very long N-terminal cytosolic domain. It contains six C2 domains (also called calcium sensors), which are spread the length of the protein and are involved in fixing calcium and phospholipids, such as phosphoinositides (14). Dysferlin also contains two specific central domains (dysferlin domains), the function of which is as yet unclear.

Studies show that dysferlin is a membrane protein in adult skeletal muscle fibres and is expressed from the 5^{th}-6^{th} week of embryonic development (2).

Dysferlin belongs to the ferlin multiprotein family, several members of which, including myoferlin, have been identified. They all have structural similarities and in particular contain C2 domains. In addition, dysferlin is highly conserved in mammals (5).

Earlier studies have shown that dysferlin interacts with various proteins such as:
caveolin-3 (12), mutations of which are responsible for LGMDIC (OMIM 607801):
calpain-3 (8), the gene mutated in LGMD2A (OMIM 253600),
annexins I and II (10),
affixin (beta-parvin), a novel integrin-linked kinase binding protein (13) and
the dihydropyridine receptor (1),
ahnak (20).

In adult skeletal muscle fibres, dysferlin plays a key role in repairing the sarcolemma, as observed in human and murine muscles deficient in dysferlin (3).

In addition, under-regulation of the complement inhibitor, decay-accelerating factor/CD55, has been observed, through analysis of messenger expression profiles, in human and murine skeletal muscles deficient in dysferlin. In vitro, the absence of CD55 leads to human myotubes becoming more susceptible to attack by complement (18).

It is crucial to resolve the question of treating dysferlinopathies.

Considering the recessive nature of dysferlinopathies, gene transfer is a possible therapeutic strategy.

The best vectors at the present time for transfecting the muscle are derived from adeno-associated viruses (AAV). However, the size of the dysferlin cDNA prevents it being directly incorporated into an AAV vector.

There is therefore a need to develop new gene therapy tools for treating dysferlinopathies.

### OBJECT OF THE INVENTION

In a first embodiment, this invention concerns a composition comprised of recombinant adeno-associated viral (AAV) vectors, preferably two in number, carrying complementary constructs allowing functional dysferlin to be expressed.

According to the invention, a first AAV vector consists of:
i) an AAV 5′ITR (Inverted Terminal Repeat) sequence;
ii) a gene portion controlled by a promoter;
iii) a sequence containing a splice donor site;
iv) an AAV 3′ITR sequence.

In addition, the second AAV vector consists of:
v) an AAV 5′ITR (Inverted Terminal Repeat) sequence;
vi) a sequence containing a splice acceptor site;
vii) a gene portion;
viii) an AAV 3′ITR sequence.

These two AAV vectors, which can be found in a single composition or in two distinct compositions, have complementary sequences which will form a functional unit at the time of concatemerisation. This occurs as an intermediary, called a concatemer, which has already been isolated and characterised in earlier techniques.

Concatemerisation occurs by the recognition of inverted terminal repeat sequences, known as ITRs, present and correctly orientated on each of the AAV vectors. Intermolecular recombination of the AAV genomes thus occurs.

The AAV ITRs are T-shaped hairpin loop structures. These sequences are essential for replication of the AAV genome, and replication and packaging of viral particles. According to the invention and in a well-known manner, vectors with non-homologous ITRs are chosen to encourage concatemerisation so that intermolecular recombination is directed towards the adjacent association of complementary heterodimers.

In addition, according to the invention, the reunited gene portions carried by the first and second AAV vectors include an open reading frame that codes for a functional to dysferlin. For this invention, “functional dysferlin” means a therapeutic protein for treating dysferlinopathies. Such a protein must satisfy, in particular, the membrane repair test described by Bansal et al. (21), and to even greater advantage, the muscle function test, including evaluation of activity time, distance covered and average speed, described in this application. It may, of course, be the complete native protein (comprised of 55 exons), in particular human, but also a mini-dysferlin (lacking certain repetitive motifs) or a mutated dysferlin retaining therapeutic activity.

To advantage, and to limit the constraint of the size of the AAV packaging, the gene portions correspond to exons. In other words, the gene portions are preferably cDNA fragments.

To advantage therefore exons 1 to 55 of human dysferlin are amplified from a vector registered in GenBank under the number NM_{—}003494.

To advantage, the reading frame formed from combining the two AAVs encodes the SEQ ID 16 sequence (AN 075923) corresponding particularly to human dysferlin described in publications (5) and (11), or to a derivative or an active fragment of it. More accurately, derivative or fragment means a protein sequence which is at least 60%, preferably 70%, still more preferably 80% or even 90% identical to the SEQ ID 16 sequence. This therefore means dysferlins of different origin (non-human mammals etc.) and mini-dysferlins.

To obtain a functional protein, to advantage the first vector carries the 5′ part of the gene, while the second vector carries the 3′ part of the same gene.

In practice, as dysferlin is composed of 55 exons, it has been determined by the inventors that, in order to have portions of this gene compatible with the size of the AAV packaging, cleavage must occur between exons 18 and 41 of the dysferlin.

The first AAV vector contains to advantage the portion of the human dysferlin gene from exon 1 to exon x, whereas the second AAV vector contains the portion from exon (x+1) to exon 55, with x being between 18 and 41. Still more advantageously, the first AAV vector contains exons 1 to 28 and the second AAV vector contains exons 29 to 55 of the human dysferlin gene.

To ensure expression of the dysferlin gene after concatemerisation, the 5′ portion of the gene, i.e. the portion carried by the first AAV vector, is placed under the control of a functional promoter. Et may for example be the c5-12 synthetic promoter, well known to those skilled in the art and specifically adapted to muscle expression of genes. Alternatively, it may be the pDesmin promoter, a derivative of the gene promoter encoding desmin.

According to a second characteristic of the invention, the sequence with the splice donor site combined with the sequence with the splice acceptor site contains all the elements necessary for splicing. In addition, these elements come to advantage from a native intron of the dysferlin gene.

To ensure this splicing, in a conventional manner, one of the vectors according to the invention provides a splice donor site and the other, a splice acceptor site.

In the context of the invention the inventors showed that, surprisingly, in the case of dysferlin, the use of native introns was not only feasible but effective.

The advantage of using native and endogenous sequences is clear: constructs can be obtained a great deal more simply and do not require the great mutagenesis envisaged in earlier techniques. In addition, for clinical applications and safety in gene therapy, it is preferable to use endogenous sequences rather than introduce exogenous sequences that could cause reactions in the host.

Thus, according to the principle of the invention, a native intron is simply “cut” into two fragments, the part including the splice donor site being naturally located after the 5′ part of the gene fragment in the first AAV vector and the part including the splice acceptor site being naturally before the 3′ part of the gene fragment in the second AAV vector.

In an optimised manner, the joining point of the sequence with the splice donor site and the sequence with the splice acceptor site corresponds to a native intron of the human dysferlin gene, to advantage one of the introns selected from introns 18 to 40, and preferably intron 28 (SEQ ID 12). This is the case for example when there is cleavage between nucleotides 140 and 141 of this intron 28.

Evidently, the sequences containing the splice donor and acceptor sites cannot be strictly identical to the native intron owing, in particular, to deletions or substitutions related to their cloning in the AAV vectors according to the invention.

This is the reason why the concept according to the invention focuses on the fact that the elements necessary for splicing, namely the splice donor and acceptor sites, the branch points and also the ESE (exonic splicing enhancer) sequences are conserved.

In a general way and for dysferlin, at least 70% of the sequence of the introns targeted, to advantage introns 18 to 40 of the human gene and preferably intron 28, is potentially involved in splicing.

To advantage, combining the sequence including the splice donor site with the sequence including the splice acceptor site produces a result which is therefore at least 70%, preferably 80%, more preferably 90% or even 95% identical to the native intron sequence.

In the particular case of intron 28, combining these two sequences produces the sequence SEQ ID 13. The latter is 97% identical to the native sequence of intron naturally located between exons 28 and 29 (SEQ ID 12).

In this specific case and when cleavage occurs after nucleotide 140 of intron 28, as the sequence including a splice donor site, the first AAV vector has the sequence SEQ ID 14, while as the sequence including a splice acceptor site, the second AAV vector has the sequence SEQ ID 15.

To advantage and to ensure good stability of the transcript, after the portion of the dysferlin gene the second AAV vector carries a polyadenylation signal, for example, the SV40 polyA.

The two vectors may be used to produce dysferlin in cells in vitro. To advantage, these cells are muscle cells, more advantageously from mammals, in particular of human origin.

An alternative to the direct use of AAV vectors is to use AAV type plasmids which include the elements i) to iv) or v) to viii) as previously defined, and thus ITR sequences, but which lack Cap and Rep coding sequences.

In another embodiment, the invention also therefore concerns a method of producing dysferlin in vitro in cells, consisting of putting the cell in contact with a composition containing the recombinant AAV vectors or plasmids according to the invention.

In practice, the two vectors or plasmids are introduced simultaneously or consecutively into the cells, particularly by transfection.

For the production of dysferlin from AAV type plasmids as described, it is also necessary to provide the Rep-Cap proteins and an “adenovirus helper” function, either as two additional plasmids during transfection or by using cell lines stably containing these sequences.

The conditions of the cell culture are adjusted by those skilled in the art so that concatemerisation, splicing and expression of the dysferlin gene occur: maintaining the vectors or plasmids, the activity of the promoter, etc.

The recombinant AAV vectors as described in this invention have obvious applications, particularly in the area of therapeutics.

Thus, another aspect of the invention concerns the use of a composition consisting of one or two AAV vectors as defined above as a medicinal product.

The corresponding pharmaceutical composition or compositions include the AAV vector or vectors, combined with a pharmaceutically acceptable inert vehicle. Various excipients, stabilisers and other suitable compounds known to those skilled in the art can be envisaged in such a composition.

When the composition according to the invention is to be injected into diseased muscles, it will preferably be in liquid form. Determining the vector concentration, the amount to be injected and the frequency of injections is part of normal practice for those skilled in the art.

Another preferred method of administration according to the invention is systemic administration.

Such medicinal products are notably intended for gene therapy, particularly for the treatment of dysferlinopathies.

As already stated, the two vectors according to the invention can be packaged in the same medicinal product, or alternatively may be in the form of two separate medicinal products.

This invention thus provides a therapeutic solution for the treatment of dysferlinopathies with the advantage of being simple, safe and effective.

### EXAMPLES OF EMBODIMENTS

The invention and the advantages resulting from it are better illustrated by the following examples of embodiments and the attached figures. These are nevertheless in no way limiting.

This example thus concerns the use of two AAV vectors capable of concatemerisation, allowing therapeutic production of a functional dysferlin.

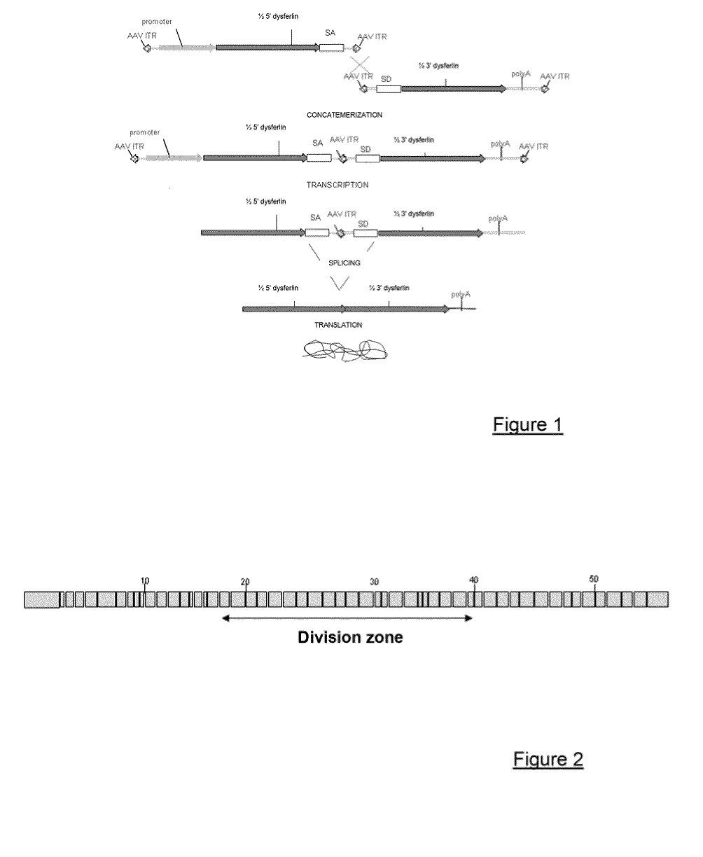

The strategy used is shown in FIG. 1. Two independent AAV vectors are used, one carrying the 5′ part of the cDNA with an intronic sequence containing a splice donor site and the other carrying an intronic sequence with a splice acceptor site followed by the 3′ part of the cDNA. In this strategy, protein expression is obtained after co-administration of the two vectors, concatemerisation of the viral genomes and splicing between the two splicing sites.

This approach applied to dysferlin was tested in vivo in dysferlin deficient mice.

FIG. 1: Diagram of the strategy used by the invention:

FIG. 2: Diagram of the dysferlin cDNA and the zone compatible for cleavage. The squares represent the different exons.

FIG. 3: Strategy used to cleave and clone the two parts of the dysferlin cDNA.

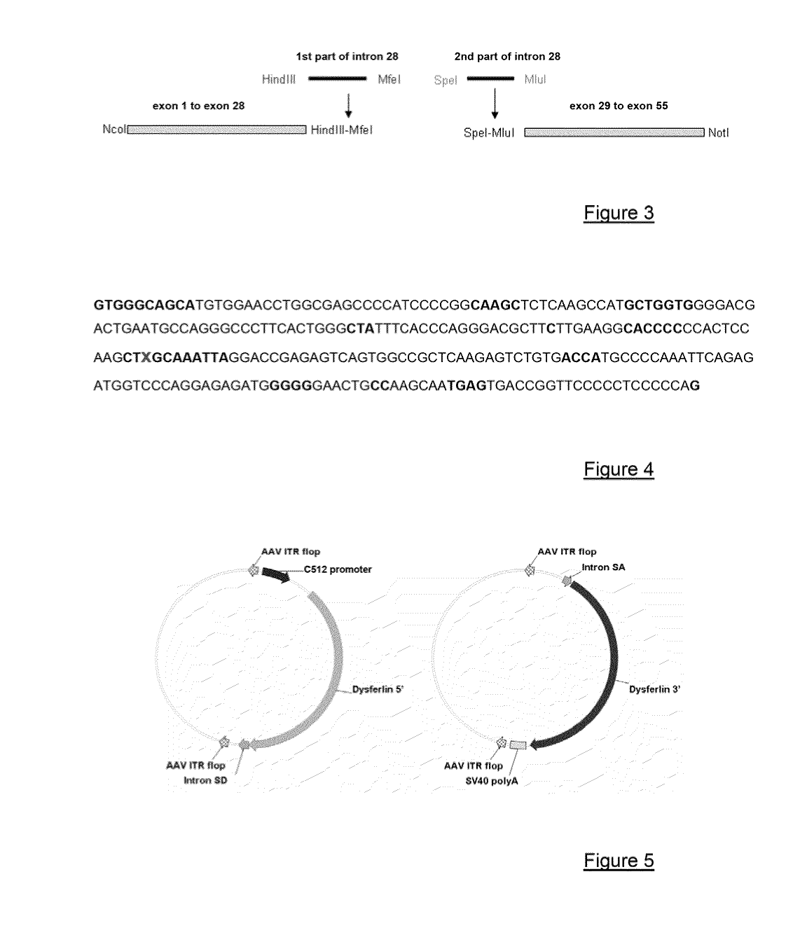

FIG. 4: Sequence of intron 28 and the position of the cleavage site within the intron (x). The nucleotides in bold are not thought to take part in ESEs.

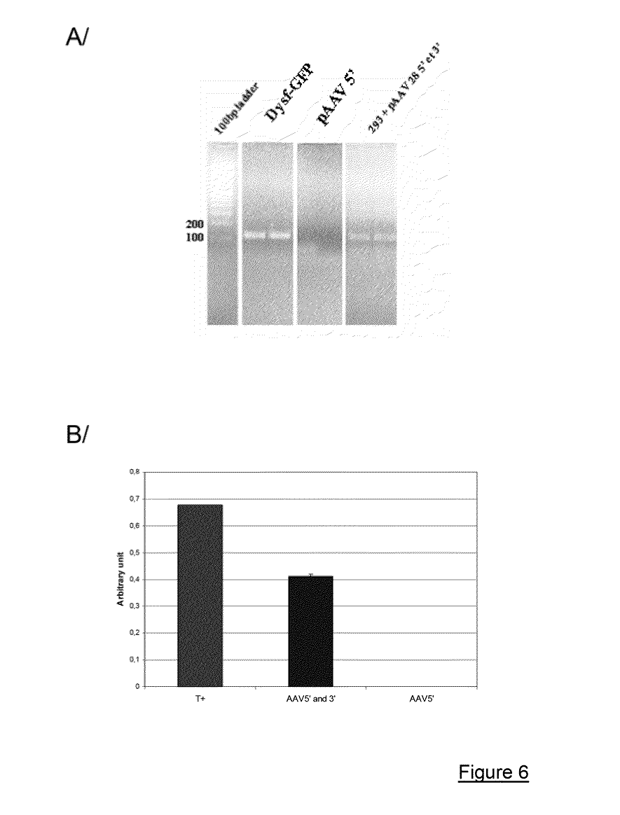

FIG. 5: Diagram of the two recombinant AAV vectors used in the invention.

FIG. 6: A/ Gel electrophoresis of the PCR product of joining. A GFP-dysferlin plasmid was used as a positive control of amplification. B/ Quantitative RT-PCR. The results are presented as the ratio of the dysferlin Ct to P0 to normalize all the samples.

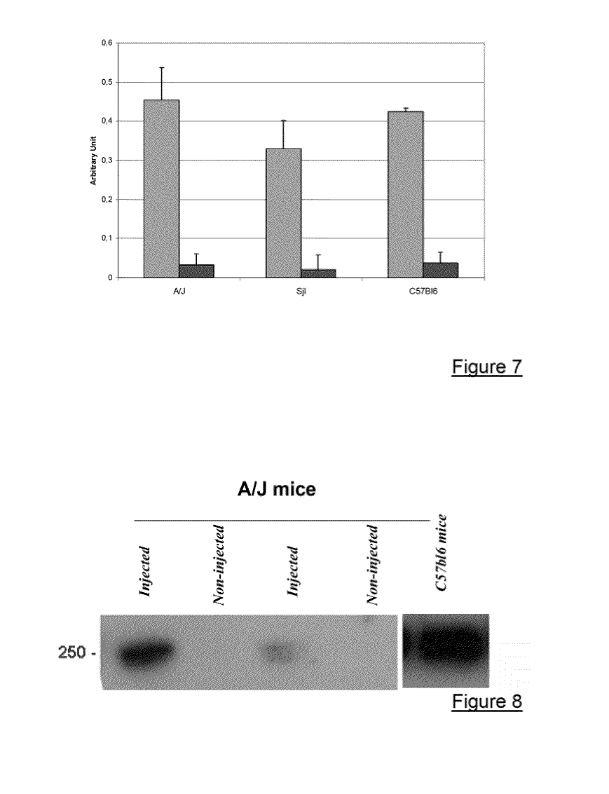

FIG. 7: RT-PCR analysis of injected muscles.

FIG. 8: Western blot analysis of injected muscles. The A/J mice are dysferlin deficient models and the C57b6 mice are normal.

FIG. 9: Measurement of the concentration of dysferlin messengers produced in the injected muscles over time.

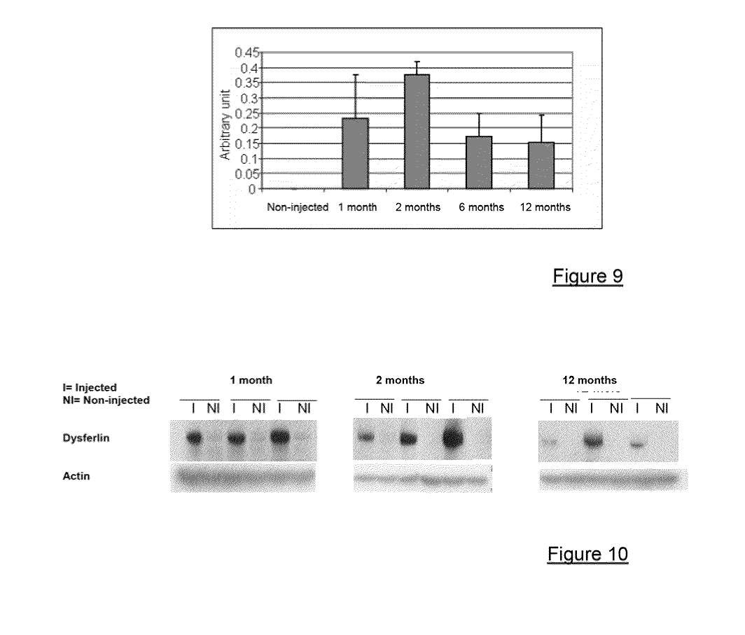

FIG. 10: Western blot detecting dysferlin protein in muscles over time. For each point, three mice were analysed.

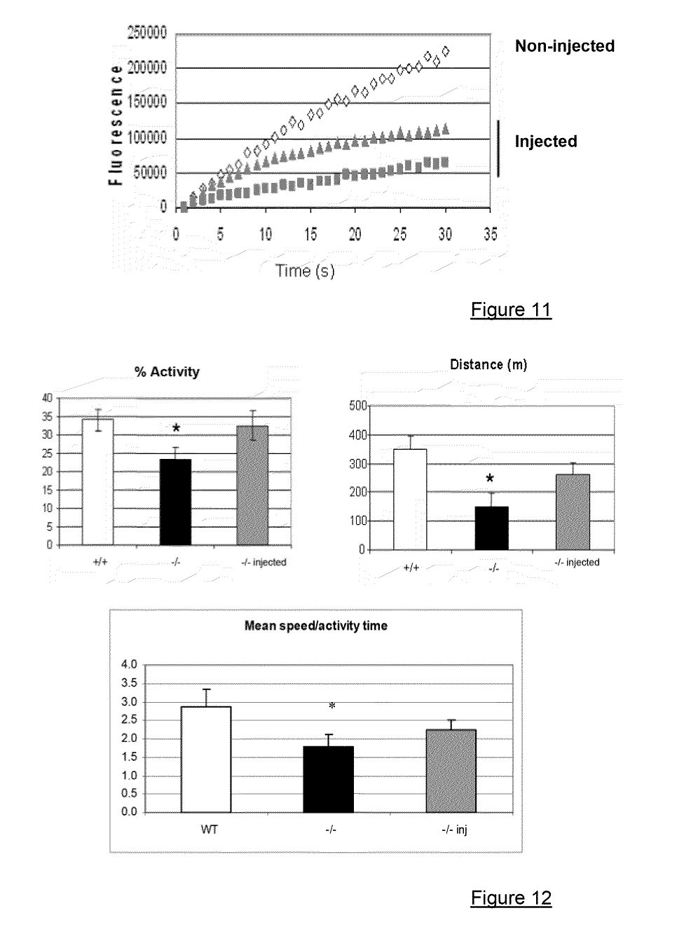

FIG. 11: Graph showing the increase in fluorescence intensity over time for muscle fibres from non-injected muscles (empty diamond) and fibres from injected muscles (shaded triangles and squares).

FIG. 12: Comparison of the % activity time, distance traveled in m and the average speed/activity time between wild-type mice (+/+), dysferlin-deficient mice (−/−) and injected mice (−/− injected).

### MATERIAL AND METHODS

### 1) Biocomputing Analyses

The following programs and web sites were used:
NNSplice: (http://www.fruitfly.org/seq_tools/splice.html);
SpliceView (http://125.itba.mi.cnr.it/˜webgene/wwwspliceview.html);
Splice Predictor (http://deepc2.psi.iastate.edu/cgi-bin/sp.cgi);
ESE-finder (http://rulai.cshl.edu/tools/ESE/), and
Rescue-ESE (http://genes.mit.edu/burgelab/rescue-ese/).

### 2) Plasmid Constructs

A pcDNA3, pGFP-Dysferlin plasmid, with the entire sequence encoding human dysferlin (GenBank number NM_{—}003494) fused to Green Fluorescent Protein (GFP) was used as the matrix for amplification of the 5′ part of dysferlin, from exon 1 to 28, using an upstream primer containing the restriction site NcoI (5′-TTCCATGGGCATGCTGAGGGTCTTCATCC-3′) (SEQ ID. 1) and a downstream primer carrying the HindIII and MfeI restriction sites (5% TTCAATTGGGAAGCTTGCCCACCTTGCTCATCGACAGCCCGG-3′) (SEQ ID 2).

This PCR product was sub-cloned in the TOPO XL PCR Cloning Kit plasmid to obtain the pTOPO-Dysf5′. The same procedure was applied to clone the 3′ part of dysferlin, from exon 29 to 55, using an upstream primer with the SpeI and MluI restriction sites (5′-TTACTAGTGGACGCGTCCAGGCTGGGAGTATAGCATCACC-3′) (SEQ ID 3) and a downstream primer carrying the restriction site NotI (5′-TTGCGGCCGCCTACAGGGCAGGAGAGTCCTCAGCTGAAGGGCTTC-3′) (SEQ ID 4) to obtain the pTOPO-dysf3′.

After digestion with the corresponding restriction enzymes (NcoI/MfeI for the 5′ part and SpeI/NotI for the 3′ part of the dysferlin), the two vectors were cloned independently in an AAV vector based on pSMD2, derived from a vector carrying type 2 ITRs (Snyder, 1997) to obtain the pAAV-dysf5′ and pAAV-dysf3′. The 5′ part was placed under the control of a C5-12 promoter (Li, 1999) and the 3′ part was followed by a polyadenylation signal from SV40.

We then used a PCR approach to insert the splice donor site sequence (SD) or the splice acceptor site sequence (SA) of the 28^{th} intron of the dysferlin gene into these two plasmids. The primers HindIII-SD5′ (5′-TTAAGCTTAGCATGTGGAACCTGG-3′ (SEQ ID 5)) and MfeI-SD3′ (5′-TTCAATTGAGCTTGGAGTGGGGGGTGC-3′ (SEQ ID 6)) were used to amplify the 5′ part of intron 28 from human genomic DNA and SpeI-SA5′ (5′-TTACTAGTGCAAATTAGGACCGAGAGTCAG-3′ (SEQ ID 7)) and MluI-SA (5′-TTACGCGTGGGAGGGGGAACCGGTCACT-3′ (SEQ ID 8)) the 3′ part of intron 28. After sufficient digestion of the plasmids and PCR products, these products were introduced in pAAV-dysf5′ and pAAV-dysf3′ to generate the pAAV2-Dysf.E28I28 and pAAV2-Dysf.I28E29 plasmids.

### 3) Production of Recombinant AAV (rAAV)

The AAV2/1 adenoviral preparations were generated by incorporating the AAV2-ITR type recombinant viral genomes into the AAV1 capsids using a plasmid tri-transfection protocol as described (4). Briefly, HEK 293 cells (60% confluence) were co-transfected with pAAV-DysfE28I28 or pAAV-DysfI28E29, the RepCap plasmid (pLT-RCO2), and the adenoviral helper plasmid (pXX6) in a 1:1:2 ratio. The crude viral lysate was harvested 60 hours after the transfection. To facilitate the release of viral particles, the crude lysate was treated sequentially by four freezing-thawing cycles, digested by benzonase (15′ at 37° C.) and precipitated with ammonium sulphate. Finally, the viral lysate was purified by two cycles of ultracentrifugation in CsCl, and then by dialysis to remove the CsCl. The viral titre was determined by real time PCR (as described in Fougerousse et al (7)).

### 4) Cell Cultures and Transfections

The HEK 293 cells were used for in vitro analysis of concatemerisation. The cells were cultured in DMEM (Dulbecco's modified Eagle medium) concentrated in glucose with the addition of 10% FCS (foetal calf serum) and 1% penicillin-streptomycin. The cells were seeded into 10 cm dishes the day before transfection. Immediately before transfection, the cells were washed with a medium of 1% FCS. The HEK 293 cells were co-transfected with pAAV-Dysf 5′, pAAV-Dysf 3′ and pLT-RCO2 in a 1:1:1 ratio. Six to seven hours after transfection, DMEM (1 g/l glucose) with 10% FCS was added. The cell cultures were collected for analysis of transgene expression 48 hours after transfection.

### 5) RT-PCR

The total RNAs were extracted from the cells by the Trizol method (Invitrogen). Residual DNA was eliminated from the samples with the DNA-free kit (Ambion). 1 μg of RNA was retrotranscribed using random primers according to the protocol of the Superscript II First Strand Synthesis system for RT-PCR (Invitrogen). The quantitative RT-PCR analyses were performed as described previously (4).

The pairs of primers and the TaqMan probe used for specific detection of spliced dysferlin were as follows: Exon28.f _{5′}CTCAACCGGGCTGTCGAT_{3′} (SEQ ID 9), Exon29.r _{5′}GTCGGTGTGTGTAGTACATCTTCTCA_{3′} (SEQ ID 10), and Exons2829.s _{5′}CAAGGCTGGGAG₃′ (SEQ ID 11). The probe (Exons2829.s) was chosen to overlap the junction between exons 28 and 29. The ubiquitous acidic ribosomal phosphoprotein (P0) was used to normalize the data between samples (4).

### 6) In Vivo Experiments

The SjI, A/J and Swiss mice came from Charles River Laboratories (Les Oncins, France). All experiments were conducted in accordance with the European Charter for the use of animals for experimental purposes. The AAVs were injected into the tibialis anterior muscle.

The mice received intramuscular injections into the left tibialis anterior (TA) of 30 μl of AAVr2/1-dysf2728 (9×10^{e}10 vg) and into the right TA, injections of 30 μl of a to mixture of AAVr2/1-dysf 2828′ and 2829 vectors in equal proportions (9×10^{e}10 vg of each). One month after injection, the mice were sacrificed and both TA muscles were removed and rapidly frozen in isopentane cooled in liquid nitrogen.

### 7) Western Blot

The muscles were homogenized using an Ultra-Turrax T8 (Ika) in lysis buffer (20 mM Tris pH 7.5, 150 mM NaCl, 2 mM EGTA, 0.1% Triton 100X; 25 μl per mg of tissue) supplemented with Complete Mini Protease Inhibitor Cocktail (Roche) and 2 μM E64 (Sigma). The samples were mixed with loading buffer [NuPage LDS (Invitrogen), 3M DTT (Sigma)], denatured for 10′ at 70° C. and rapidly centrifuged. The samples were loaded onto NuPage precast 3-10% polyacrylamide gradient gels (Invitrogen). The proteins were separated by electrophoresis in MOPS buffer, then they were electro-transferred (100V for one hour) onto a PVDF membrane (Immobilon-P PVDF transfer membrane, Dutscher). The efficacy of the transfer was verified by Ponceau red staining (0.2% Ponceau red/1% acetic acid), followed by decolourising in 1% acetic acid. The membranes were then left for one hour at ambient temperature with 3% bovine serum albumin (BSA) in Tris saline buffer with 0.1% Tween-20 (TTBS) and hybridised with primary murine monoclonal antibodies against dysferlin (NCL-Hamlet, Novocastra, dilution 1:500) at ambient temperature for ⅔ hours. Finally, the membranes were incubated for one hour with horseradish peroxidase (HRP) conjugated secondary anti-mouse antibody (1:1,000 in TTBS) (Amersham Biosciences, Piscataway, N.J., USA). Detection was performed with the SuperSignal West Pico Chemiluminescent Substrate Kit (Pierce, Rockford, Ill., USA). Specific bands were visualised by exposing the membranes on X-OMAT-S films (Hyperfilm ECL, Amersham Biosciences).

### II) RESULTS

This strategy uses two independent AAV vectors, one carrying the 5′ part of the cDNA with an intronic sequence containing a splice donor site and the other carrying an intronic sequence with a splice acceptor site followed by the 3′ part of the cDNA. In this strategy, protein expression is obtained after co-administration of the two vectors, concatemerisation of the viral genomes and splicing between the two splice sites (FIG. 1). This approach has been tested in vivo in dysferlin deficient mice.

1) Construction of AAV Vectors

The 6.2 kb of the dysferlin messenger are encoded by 55 exons. Considering the packaging capacity of AAV vectors and the minimum size of the necessary regulator elements, it is theoretically possible to divide the dysferlin cDNA between exons 18 and 41 (FIG. 2).

For this invention it was decided to use endogenous sequences of the dystrophin gene as the splicing elements to be introduced into both vectors to allow splicing of the two parties of the dysferlin cDNA.

For this embodiment, the sequence encoding dysferlin was cleaved in the 28^{th} intron (FIG. 3). The sequence of this intron (SEQ ID 12) is shown in FIG. 4. This intron is small (264 bp) and can be used in its entirety, ensuring that all necessary signals for splicing are present. It was thus possible to verify that the donor and acceptor splicing sites scored correctly using different programs (NNSplice; SpliceView; Splice Preditor; Table 1). The cleavage site is indicated by a cross in the sequence shown in FIG. 4 (between nucleotides 140 and 141).

TABLE 1
Intron 28 splice site scores

| | Splice donor site | | Splice acceptor site | | |
|---|---|---|---|---|---|
| NNSplice | SpliceView | SplicePredictor | NNSplice | SpliceView | SplicePredictor |
| 0.77 | 0.86 | 0.84 | 0.89 | 0.91 | X |

Exon splicing enhancers (ESEs) were subsequently sought in this intron, using ESE-finder and Rescue-ESE to avoid inserting ITR sequences in regions that regulate splicing. Nucleotides not involved in ESEs according to the programs used are shown in bold in FIG. 4.

To construct the desired vectors, the cDNA halves and half introns were amplified using primers with restriction sites, and then successively inserted into the AAV vectors to obtain the pAAV-Dysf5′-E28I28 and pAAV-I28E29Dysf3′ (FIG. 5).

2) Expression of the Complete Human Dysferlin in Cells

293 cells were quadri-transfected with pAAV-Dysf5′-E28I28, pAAV-I28E29Dysf3′ and RepCap and adenovirus helper plasmids. Transfection with pAAV-Dysf5′-E28I28 alone was also carried out, to act as a control. After RNA extraction, a quantitative RT-PCR was performed to detect human dysferlin expression with primers flanking the junction of the AAV concatemer. As expected, no dysferlin expression was detected in 293 cells transfected with the vector 5′ alone, while a band of the expected size was detected in cells transfected with both vectors (FIG. 6).

3) Expression of Complete Human Dysferlin after Intramuscular Injection in Mice.

Having validated the possibility of obtaining a complete human dysferlin messenger from the two vectors, we looked to see whether a similar event could occur in vivo in the muscles of mice. We injected 9×10¹⁰ viral genomes (vg) of each vector into the tibialis anterior muscle (TA) of three strains of mice, normal, SJL and A/J dysferlin deficient, aged 4 months. The muscles were removed 35 days after injection and analysed for expression of the level of messengers by quantitative TaqMan. As FIG. 7 shows, a substantial level of transcript was obtained (FIG. 7).

The efficacy of gene transfer was assessed by Western blot. Analysis of the injected TA showed expression of the complete protein at 250 kDa (FIG. 8).

4) Effect of Intramuscular Injection Over Time

In order to analyse expression of the transgene with time, 4/5-month-old A/J (dysferlin-deficient) mice received injections into the tibialis anterior muscle (TA). The muscle of the left paw received the two vectors AAV2/1-Dysf5′-E28I28 and AAV2/1-I28E29Dysf3′ (9×10e10 viral genomes (vg) of each) and the muscle of the right paw the vector AAV 5′, AAV2/1-Dysf5′-E28I28 (1.5×10e11 vg) only. Mice were sacrificed 1, 2, 6 or 12 months after injection.

The muscles were removed and the level of mRNA and transgenic proteins analysed. The level of mRNA was quantified by quantitative RT-PCR (qRT-PCR). At each point in time TaqMan showed a significant level of dysferlin in the muscles injected with both vectors (FIG. 9). On the other hand, no messenger was detected in the contralateral muscle.

Western blot analysis of the muscles injected revealed that dysferlin was detected at a size corresponding to the whole protein (237 kDa), while no band was detected in the contralateral non-injected muscles (FIG. 10).

These results indicate that the two vectors injected produce long-term expression of the complete messenger and of the dysferlin protein.

5) Evaluation of Membrane Repair

In a test based on producing lesions in the plasma membrane of muscle fibres with a laser beam, it has been shown that dysferlin plays a role in membrane repair (21). We therefore evaluated the ability to repair muscle fibres after injection of the vectors. This experiment was conducted on the flexor digitorum brevis muscle (FDB) after intramuscular injection of 7.5×10e10 vg of each vector into 3/4-month-old dysferlin deficient mice. One month after injection, the muscles were removed and the fibres were individualised after digestion with collagenase. The latter were placed in a solution containing the dye FM 1-43 with or without calcium. Lesions were induced by maximum irradiation for one second with a two-photon laser of a confocal microscope. Images of the penetration of stain were then acquired for 3 min every 7 seconds. The intensity of fluorescence was quantified using the ImageJ program.

As expected, the fibres from deficient mice were unable to repair the lesions induced, while the same lesion was repaired efficiently in fibres from injected mice (FIG. 11).

6) Evaluation of Muscle Function after Intramuscular Injection

The basal locomotor activity of mice was quantified using an actimeter (apparatus for recording by infrared sensor how much mice move about) over a period of 6 hours. This experiment was conducted on mice injected one month earlier with 7.2×10e12 vg of each vector into the caudal vein.

The activity period of deficient mice was decreased by 31.4%. There was a reduction in the distance covered of 56.9% and a decrease of 37.24% in the average speed over the time of activity, relative to the wild type. Decreases in injected deficient mice were only 4.2%, 24.6% and 21.28% for these three parameters (FIG. 12).

### CONCLUSIONS

We have exploited the ability of AAV vectors to concatemerise to produce expression of the messenger and complete dysferlin protein. To this end, a 5′ vector was to generated carrying exons 1 to 28 of the dysferlin cDNA and half of intron 28 bearing the splice donor site, and a complementary 3′ vector on the other half of intron 28, with the splice acceptor site, followed by the rest of the dysferlin cDNA and a polyadenylation signal. Subsequently, co-transfection into 293 cells or intramuscular injections in animal models were performed; the 5′ and 3′ vectors together produced whole human dysferlin.

More precisely, the efficacy of the vectors used in this strategy to reconstruct dysferlin after intramuscular or intravascular injection in a mouse model of LGMD2B has been demonstrated by the stable expression of the whole dysferlin protein. In addition, expression of the transgene is associated with restoring membrane repair capacity and increased locomotor activity.

Thus, these results show the potential use of AAV concatemerisation for expression of dysferlin as a promising strategy in human dysferlin deficiency. Importantly, this has been undertaken using sequences of the endogenous dysferlin gene. This is an advantage in gene therapy where the introduction of exogenous sequences (a possible source of reactions or undesirable recombinations) is avoided to the maximum and simplifies obtaining the constructs. Compensation for a lack of dysferlin has therefore been obtained simply and effectively by gene therapy.

### BIBLIOGRAPHY

(1) Ampong, B. N., M. Imamura, T. Matsumiya, M. Yoshida, and S. Takeda. 2005. Intracellular localization of dysferlin and its association with the dihydropyridine receptor. Acta Myol. 24:134-44.
(2) Anderson, L. V., K. Davison, J. A. Moss, C. Young, M. J. Cullen, J. Walsh, M. A. Johnson, R. Bashir, S. Britton, S. Keers, Z. Argov, I. Mahjneh, F. Fougerousse, J. S. Beckmann, and K. M. Bushby. 1999. Dysferlin is a plasma membrane protein and is expressed early in human development. Hum Mol Genet. 8:855-61.
(3) Bansal, D., K. Miyake, S. S. Vogel, S. Groh, C. C. Chen, R. Williamson, P. L. McNeil, and K. P. Campbell. 2003. Defective membrane repair in dysferlin-deficient muscular dystrophy. Nature. 423:168-72.
(4) Bartoli, M., J. Poupiot, A. Goyenvalle, N. Perez, L. Garcia, O. Danos, and I. Richard. 2006. Noninvasive monitoring of therapeutic gene transfer in animal models of muscular dystrophies. Gene Ther. 13:20-8.
(5) Bashir, R., S. Britton, T. Strachan, S. Keers, E. Vafiadaki, M. Lako, I. Richard, S. Marchand, N. Bourg, Z. Argov, M. Sadeh, I. Mahjneh, G. Marconi, M. R. Passos-Bueno, S. Moreira Ede, M. Zatz, J. S. Beckmann, and K. Bushby. 1998. A gene related to Caenorhabditis elegans spermatogenesis factor fer-1 is mutated in limb-girdle muscular dystrophy type 2B. Nat Genet. 20:37-42.
(6) Cagliani, R., F. Magri, A. Toscano, L. Merlini, F. Fortunato, C. Lamperti, C. Rodolico, A. Prelle, M. Sironi, M. Aguennouz, P. Ciscato, A. Uncini, M. Moggio, N. Bresolin, and G. P. Comi. 2005. Mutation finding in patients with dysferlin deficiency and role of the dysferlin interacting proteins annexin A1 and A2 in muscular dystrophies. Hum Mutat. 26:283.
(7) Fougerousse, F., Bartoli, M., Poupiot, J., Arandel, L., Durand, M., Danos, O., Richard, I. 2007. Phenotypic correction of α-sarcoglycan deficiency by intra-arterial injection of a muscle specific AAV1 serotype vector. Molecular Therapy. Sous presse
(8) Huang, Y., P. Verheesen, A. Roussis, W. Frankhuizen, I. Ginjaar, F. Haldane, S. Laval, L. V. Anderson, T. Verrips, R. R. Frants, H. de Haard, K. Bushby, J. den Dunnen, and S. M. van der Maarel. 2005. Protein studies in dysferlinopathy patients using llama-derived antibody fragments selected by phage display. Eur J Hum Genet. 13:721-30.
(9) Illa, I., C. Serrano-Munuera, E. Gallardo, A. Lasa, R. Rojas-Garcia, J. Palmer, P. Gallano, M. Baiget, C. Matsuda, and R. H. Brown. 2001. Distal anterior compartment myopathy: a dysferlin mutation causing a new muscular dystrophy phenotype. Ann Neurol. 49:130-4.
(10) Lennon, N. J., A. Kho, B. J. Bacskai, S. L. Perlmutter, B. T. Hyman, and R. H. Brown, Jr. 2003. Dysferlin interacts with annexins A1 and A2 and mediates sarcolemmal wound-healing. J Biol Chem. 278:50466-73.
(11) Liu, J., M. Aoki, I. Illa, C. Wu, M. Fardeau, C. Angelini, C. Serrano, J. A. Urtizberea, F. Hentati, M. B. Hamida, S. Bohlega, E. J. Culper, A. A. Amato, K. Bossie, J. Oeltjen, K. Bejaoui, D. McKenna-Yasek, B. A. Hosler, E. Schurr, K. Arahata, P. J. de Jong, and R. H. Brown, Jr. 1998. Dysferlin, a novel skeletal muscle gene, is mutated in Miyoshi myopathy and limb girdle muscular dystrophy. Nat Genet. 20:31-6.
(12) Matsuda, C., Y. K. Hayashi, M. Ogawa, M. Aoki, K. Murayama, I. Nishino, I. Nonaka, K. Arahata, and R. H. Brown, Jr. 2001. The sarcolemmal proteins dysferlin and caveolin-3 interact in skeletal muscle. Hum Mol Genet. 10:1761-6.
(13) Matsuda, C., K. Kameyama, K. Tagawa, M. Ogawa, A. Suzuki, S. Yamaji, H. Okamoto, I. Nishino, and Y. K. Hayashi. 2005. Dysferlin interacts with affixin (beta-parvin) at the sarcolemma. J Neuropathol Exp Neurol. 64:334-40.
(14). Nalefski, F. A., and J. J. Falke. 1996. The C2 domain calcium-binding motif: structural and functional diversity. Protein Sci. 5:2375-90.
(15) Nguyen, K., G. Bassez, R. Bernard, M. Krahn, V. Labelle, D. Figarella-Branger, J. Pouget, H. Hammouda el, C. Beroud, A. Urtizberea, B. Eymard, F. Leturcq, and N. Levy. 2005. Dysferlin mutations in LGMD2B, Miyoshi myopathy, and atypical dysferlinopathies. Hum Mutat. 26:165.
(16) Tahara, M., R. G. Pergolizzi, H. Kobayashi, A. Krause, K. Luettich, M. L. Lesser, and R. G. Crystal. 2004. Trans-splicing repair of CD40 ligand deficiency results in naturally regulated correction of a mouse model of hyper-IgM X-linked immunodeficiency. Nat Med. 10:835-41.
(17) Ueyama, H., T. Kumamoto, H. Horinouchi, S. Fujimoto, H. Aono, and T. Tsuda. 2002. Clinical heterogeneity in dysferlinopathy. Intern Med. 41:532-6.
(18) Wenzel, K., J. Zabojszcza, M. Carl, S. Taubert, A. Lass, C. L. Harris, M. Ho, H. Schulz, O. Hummel, N. Hubner, K. J. Osterziel, and S. Spuler. 2005. Increased susceptibility to complement attack due to down-regulation of decay-accelerating factor/CD55 in dysferlin-deficient muscular dystrophy. J Immunol. 175:6219-25.
(19) Xu, Z., Y. Yue, Y. Lai, C. Ye, J. Qiu, D. J. Pintel, and D. Duan. 2004. Trans-splicing adeno-associated viral vector-mediated gene therapy is limited by the accumulation of spliced mRNA but not by dual vector coinfection efficiency. HUM Gene Ther. 15:896-905.
(20) Huang Y, Laval S H, van Remoortere A, Baudier J, Benaud C, Anderson L V, Straub V, Deelder A, Frants R R, den Dunnen J T, Bushby K, van der Maarel S M. 2007. AHNAK, a novel component of the dysferlin protein complex, redistributes to the cytoplasm with dysferlin during skeletal muscle regeneration. FASEB Journal 21(3):732-42.
(21) Bansal, D., K. Miyake, S. S. Vogel, S. Groh, C. C. Chen, R. Williamson, P. L. McNeil, and K. P. Campbell. 2003. Defective membrane repair in dysferlin-deficient muscular dystrophy. Nature 423:168-72.

1/ Composition including at least:
a first adeno-associated viral (AAV) vector including:
i) an AAV 5′ITR (Inverted Terminal Repeat) sequence;
ii) a gene portion controlled by a promoter;
iii) a sequence containing a splice donor site;
iv) an AAV 3′ITR sequence:
and
a second adeno-associated viral (AAV) vector including:
v) an AAV 5′ITR (Inverted Terminal Repeat) sequence;
vi) a sequence containing a splice acceptor site;
vii) a gene portion;
viii) an AAV 3′ITR sequence;
characterised by:
the reunited gene portions carried by the first and second AAV vectors including an open reading frame which codes for a functional dysferlin, to advantage of human origin;
the sequence with the splice donor site combined with the sequence with the splice acceptor site containing all the elements necessary for splicing.

2/ Composition according to claim 1 characterised by all the necessary elements for splicing coming from a native intron of the dysferlin gene, advantageously of human origin.

3/ Composition according to claim 2 characterised by the sequence with the splice donor site combined with the sequence with the splice acceptor site containing all the elements necessary for splicing contained in one of introns 18 to 40 of the human dysferlin gene, to advantage in intron 28.

4/ Composition according to claim 3 characterised by the sequence with the splice donor site combined with the sequence with the splice acceptor site being at least 70% identical to the sequence of intron 28 (SEQ ID 12), and having to advantage the sequence SEQ ID 13.

5/ Composition according to one of the previous claims characterised by:
sequence iii) of the first AAV having the sequence SEQ ID 14;
sequence vi) of the second AAV having the sequence SEQ ID 15.

6/ Composition according to one of the previous claims characterised by:
sequence ii) of the first AAV corresponding to exons 1 to x of the dysferlin gene:
sequence vii) of the second AAV corresponding to exons (x+1) to 55 of the dysferlin gene,
with x being between 18 and 41, to advantage being 28.

7/ Composition according to one of the previous claims characterised by the first AAV vector including the synthetic promoter C5-12 or the desmin promoter, functionally linked to the portion of the dysferlin gene.

8/ Composition according to one of the previous claims characterised by the second AAV vector including a polyadenylation signal, to advantage the polyA sequence of SV40, after the portion of the dysferlin gene.

9/ Composition according to one of the preceding claims as a combination composition for use simultaneously, separately or staggered over time in gene therapy, particularly for the treatment of dysferlinopathies.

10/ Method for expressing dysferlin in vitro in a host cell comprising putting into contact the cell and the composition according to one of claims 1 to 9 or the cell and the plasmids to obtain a composition according to one of claims 1 to 9.

11/ Method For expressing dysferlin according to claim 10 characterised by the host cell being a mammalian cell, preferably a human cell.

12/ Method for expressing dysferlin according to claim 10 or 11 characterised by the host cell being a muscle cell.

13/ Using the composition according to any of claims 1 to 9 for the preparation of a medicinal product.

14/ Using the composition according to any of claims 1 to 9 for the manufacture of one or more medicinal products for gene therapy, particularly for the treatment of dysferlinopathies.

15/ Use according to claim 13 or 14 characterised by the two AAV vectors being packaged in the same medicinal product.

16/ Use according to claim 13 or 14 characterised by each AAV vector being packaged in a separate medicinal product.
